(19) United States
(12) Patent Application Publication
(10) Pub. No.: US 2014/0052009 A1
(43) Pub. Date: Feb. 20, 2014

(54) MONITORING BLOOD PRESSURE IN A MEDICAL INJECTION SYSTEM

(71) Applicants: Sidney Donald Nystrom, Shoreview, MN (US); David D. Scott, Minneapolis, MN (US)

(72) Inventors: Sidney Donald Nystrom, Shoreview, MN (US); David D. Scott, Minneapolis, MN (US)

(73) Assignee: ACIST MEDICAL SYSTEMS, INC., Eden Prairie, MN (US)

(21) Appl. No.: 13/586,658

(22) Filed: Aug. 15, 2012

(51) Int. Cl. A61B 5/021; A61B 5/00; A61M 5/168; A61B 5/0215

(52) U.S. Cl. CPC A61B 5/02141; A61B 5/0215; A61B 5/4839; A61M 5/16854; A61B 5/7217
USPC 600/487

(57) ABSTRACT

A pressure sensor, for example, employed by a medical injection system for monitoring blood pressure, is isolated from relatively high pressures by a volume of a compressible medium, preferably air, or other suitable gas. The volume fills an entirety of a cavity of a pressure transducer assembly, and is enclosed between a pressure transmission interface of the assembly and the pressure sensor. The pressure transducer assembly is integrated into a fluid circuit such that the pressure transmission interface is exposed to flow through the fluid circuit. The cavity of the assembly has a configuration that allows transmission of a patient's blood pressure from the fluid circuit, via the interface and gas volume, to the pressure sensor, yet prevents transmission of the relatively high pressures, for example, injection pressures. The pressure transducer assembly may be part of a disposable fluid circuit subassembly, for example, packaged as a kit.

### TECHNICAL FIELD

The present disclosure pertains to medical injection systems and more particularly to apparatus and methods for isolating blood pressure monitoring sensors thereof.

### BACKGROUND

A medical injection system, for example, to deliver a contrast agent into a patient's vascular system for medical imaging, typically includes a pressure sensor integrated into a fluid circuit of the system for the purpose of monitoring the patient's blood pressure during the imaging procedure. However, contrast media is injected at pressures that are significantly higher (i.e. up to 1200 psi) than the blood pressure being monitored (i.e. between 0 and 5 psi), so that the typical blood pressure monitoring pressure sensor is protected from exposure to the high injection pressures, for example, by isolating that portion of the fluid circuit to which the pressure sensor is coupled from that portion through which the high pressure injection flows. One example of such a medical injection system, the ACIST CVⱼ™ system, is shown in FIG. 1.

FIG. 1 is a perspective view of an exemplary medical injection system 100 wherein a first fluid reservoir 132 supplies a pressurizing unit 130 for injection of, for example, a radiopaque contrast agent, into a patient's vascular system via a fluid circuit line 304 that feeds into another fluid circuit line 122. FIG. 1 further illustrates a second fluid reservoir 138 from which a diluent, such as saline, is drawn by a peristaltic pump 106 through yet another fluid circuit line 128 that feeds into line 122. The fluid circuit of system 100 further includes a manifold valve 124 and associated sensor 114 to control the flow of fluids into line 122, from pressurizing unit 130 and from line 128. When valve 124 is open to line 128 and closed to line 304 from pressurizing unit 130, and line 122 is coupled to the patient's vascular system, for example, by a patient line connected to line 122 at a connector 120, a pressure transducer assembly 126, which is integrated into line 128, monitors the patient's blood pressure. But, when pressurizing unit 130 is activated to inject a contrast agent, valve 124 is switched to allow the relatively high pressure flow from pressurizing unit 130 into line 122, and to isolate line 128 from the high pressure flow, not only to prevent backflow into line 128, but to also protect the pressure transducer of assembly 126 from exposure to the relatively high injection pressures that could damage the pressure sensor thereof.

One example of a pressure transducer assembly that may be employed by system 100 is the LogiCal® system available from Smiths Medical International; and another example is the Meritrans® available from Merit Medical Systems, Inc. Each of the aforementioned transducer assemblies includes a relatively low cost and disposable pressure sensor (i.e. intended for use in a single medical procedure), with an operating range that is suitable for blood pressure measurements. Thus, as alluded to above, this type of sensor would be rendered inoperable for blood pressure monitoring in between multiple injections (common in a single medical imaging procedure), if exposed to the relatively high injection pressures. Although more robust pressure sensors, which are sensitive enough for blood pressure monitoring, yet are not damaged by exposure to the higher injection pressures, are available, the cost of such sensors is prohibitive for disposable/single use medical applications.

FIG. 1 further illustrates system 100 including an air bubble detector 116, which may generate an alarm upon detection of a significant volume of air in line 122. Those skilled in the art understand that the presence of a significant volume of air in the fluid circuit not only poses the risk of creating air emboli within the patient's vascular system during injections of contrast media, but can also distort the aforementioned blood pressure monitoring by adding compliance to the fluid circuit that can effectively lower the natural frequency response of the pressure sensor.

### SUMMARY

Some embodiments and methods of the present invention are directed toward isolating a blood pressure monitoring sensor of a medical injection system from relatively high injection pressures. A pressure transducer assembly for a medical injection system, according to some embodiments and methods, is configured to enclose a volume of a compressible medium, preferably air, or other suitable gas, between a pressure sensor of the assembly and a pressure transmission interface of the assembly, for example, a flexible gas-permeable membrane. The transducer assembly may be part of a disposable fluid circuit subassembly, for example, packaged as a kit. According to preferred embodiments, the gas volume, for example, no greater than approximately ten cubic millimeters, fills an entirety of a cavity that extends between the pressure transmission interface and the pressure sensor; wherein a configuration of the cavity allows the gas volume to transmit a patient's blood pressure from the fluid circuit, via the interface, to the pressure sensor, yet prevents the volume from transmitting the relatively high, and potentially damaging, pressures of injection flow to the pressure sensor.

A ratio of a volume of a first part of the cavity of the pressure transducer assembly, which is adjacent to the pressure transmission interface, to a volume of a second part of the cavity, which is adjacent to the pressure sensor, is preferably between approximately one and approximately six, and, according to some embodiments, the second part of the cavity extends as a bore from an opening in a floor of the first part of the cavity. In preferred embodiments, the pressure transmission interface collapses, or moves into contact with the floor of the first part of the cavity in response to the relatively high injection pressures, and then rebounds out of contact when the pressure subsides.

It should be noted that embodiments and methods of the present invention will find application in other areas, besides medical injection systems, where it is desirable to employ relatively low cost, mass-produced pressure sensors for measuring/monitoring relatively low pressures without concern for exposure to relatively high pressures outside the range of the sensors. Examples of other potential medical applications include, without limitation, pressure monitoring for wound therapy machines, for hospital beds, and for oxygen concentrators, or even for other types of medical infusion devices, for example, hand manifolds.

### BRIEF DESCRIPTION OF THE DRAWINGS

The following drawings are illustrative of particular methods and embodiments of the present disclosure and, therefore, do not limit the scope of the invention. The drawings are not to scale (unless so stated) and are intended for use in conjunction with the explanations in the following detailed description. Methods and embodiments will hereinafter be described in conjunction with the appended drawings, wherein like numerals denote like elements, and:

FIG. 1 is a perspective view of an exemplary medical injection system;

FIG. 2 is a block diagram of an injection system fluid circuit with an enlarged cross-section schematic of an included pressure transducer assembly, configured according to some embodiments and methods of the present invention;

FIG. 3 is a plan view of a disposable fluid circuit subassembly, according to some embodiments;

FIG. 4 is a cross-section view through section line A-A of FIG. 3, according to some embodiments;

FIG. 4A is an enlarged cross-section view of a portion of the assembly shown in FIG. 4;

FIG. 5 is an exploded view of the pressure transducer assembly shown in FIG. 3, according to some embodiments of the present invention;

FIG. 5A is a schematic cross-section through a portion of the pressure transducer assembly, according to some alternate embodiments;

FIG. 5B is a schematic cross-section through a portion of the pressure transducer assembly, showing an optional feature, according to some embodiments;

FIG. 5C is a perspective view of another portion of the pressure transducer assembly, according to some embodiments;

FIG. 6 is a perspective view of an alternate embodiment of a portion of the pressure transducer assembly; and

FIG. 7 is a block diagram of an alternate configuration of an injection system fluid circuit with an enlarged plan view of a subassembly thereof, according to some alternate embodiments.

### DETAILED DESCRIPTION

The following detailed description is exemplary in nature and is not intended to limit the scope, applicability, or configuration of the invention in any way. Rather, the following description provides practical illustrations for implementing exemplary methods and embodiments. Examples of constructions, materials and dimensions are provided for selected elements, and all other elements employ that which is known to those of skill in the field of the invention. Those skilled in the art will recognize that many of the examples provided have suitable alternatives that can be utilized.

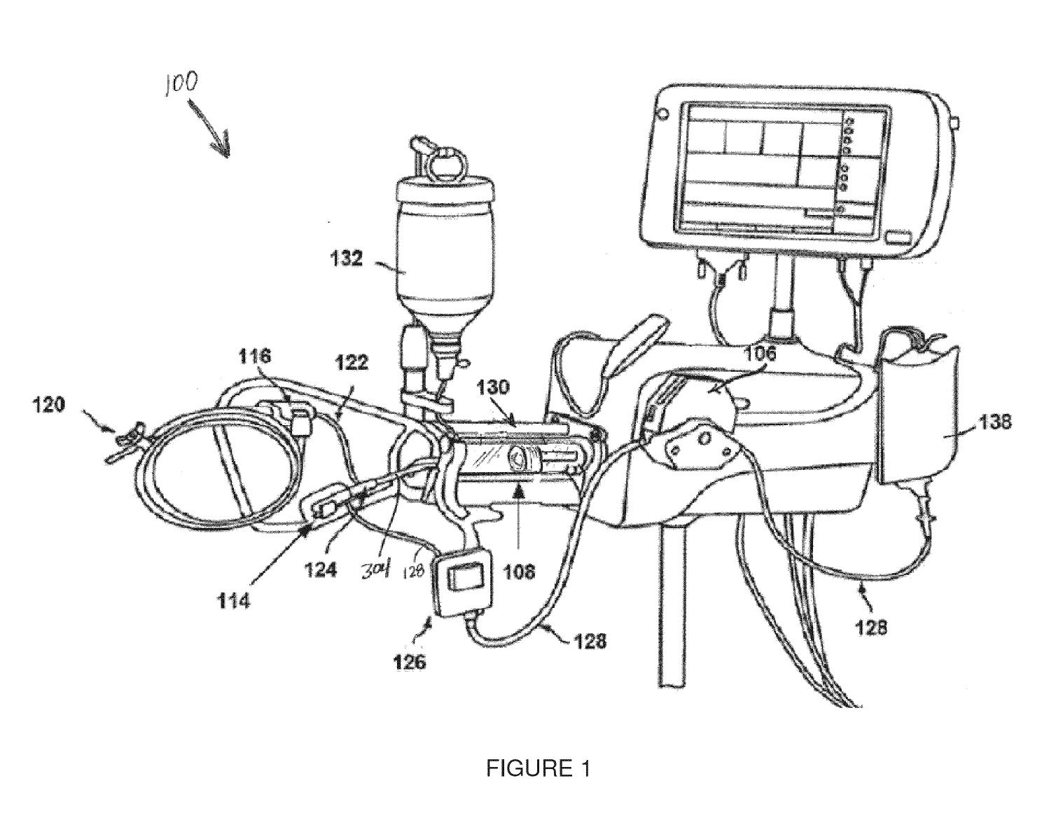

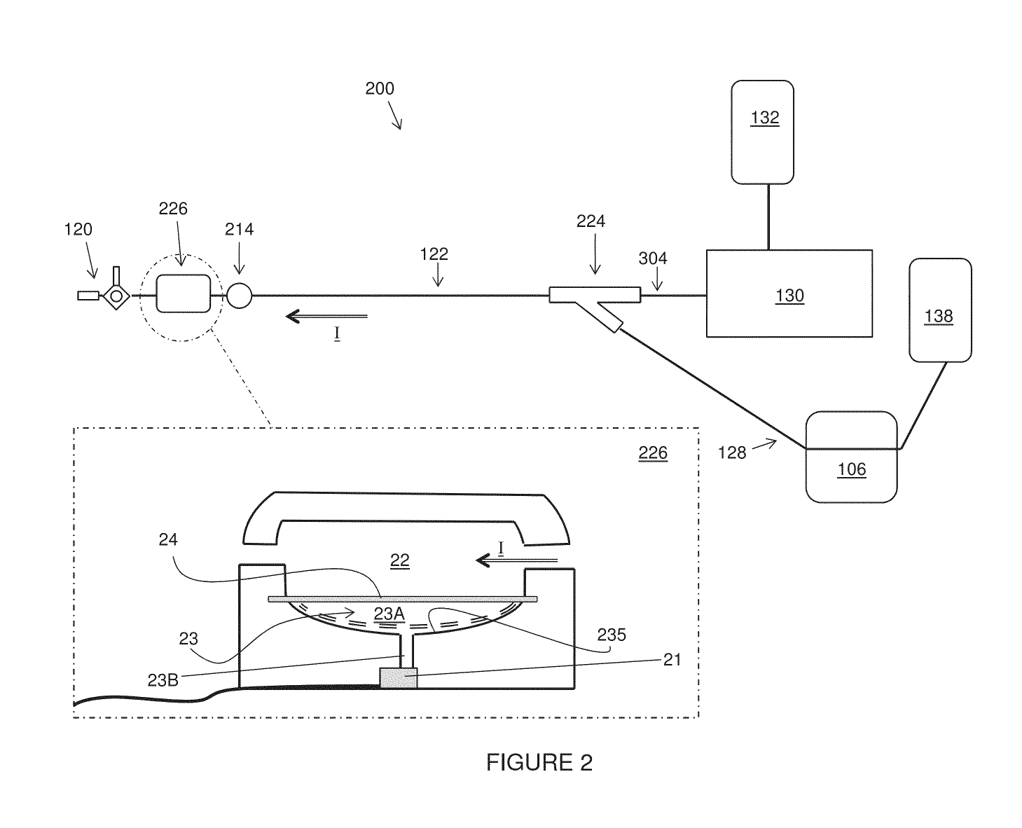

FIG. 2 is a block diagram of an injection system fluid circuit 200, along with an enlarged schematic cross-section of a pressure transducer assembly 226, configured according to some embodiments and methods, which is included therein. Fluid circuit 200 is similar to that shown for system 100 in FIG. 1, but, rather than being integrated into fluid circuit line 228 like pressure transducer assembly 126, pressure transducer assembly 226 is integrated into line 122. FIG. 2 illustrates assembly 226 being located in relatively close proximity to connector 120, to which a patient line is connected for access to the patient's vascular system, so that a relatively sensitive disposable pressure sensor 21, which is mounted therein, may operate in relatively close proximity to the patient to monitor the patient's blood pressure. A check valve 214 is shown located on an upstream side of assembly 226 to prevent backflow during blood pressure monitoring. (A direction of injection flow is indicated with an arrow I.) Monitoring blood pressure in relatively close proximity to the patient is desirable in order to minimize blood pressure signal distortions, which may be caused by mechanical factors imposed by an increased volume of fluid within fluid circuit lines that extend between the patient's vascular system and a pressure sensor. Some of these factors include: 1) fluid resistance due to friction; 2) compliance or stiffness of fluid-filled tubing lines; and 3) fluid inertance (i.e. a measure of pressure gradient in the fluid required to cause a change in flow rate with time).

According to methods of the present invention, assembly 226 incorporates a volume of air, or other suitable gas, that protects pressure sensor 21 from the relatively high pressure injection flow, through line 122, that is generated by pressurizing unit 130, yet does not degrade the blood pressure monitoring performance of pressure sensor 21 during the time between high pressure injections. FIG. 2 further illustrates assembly 226 including a cavity 23, which is entirely filled with the volume of air/gas. It should be noted that, for alternative applications, for example, in which sterilization of the pressure transducer assembly is not necessary, an alternative compressible medium, such as a gel, may be substituted for the volume of gas.

According to FIG. 2, the volume of air/gas is enclosed in cavity 23 between a first side of a pressure transmission interface 24 and pressure sensor 21. Interface 24, for example, a flexible gas-permeable membrane, extends between cavity 23 and a flow channel 22 of fluid circuit line 122 such that a second side of interface 24 is exposed to flow channel 22. Cavity 23 is shown including a first part 23A and a second part 23B, which configuration allows the gas/air volume to transmit relatively low pressures, i.e. the patient's blood pressure, from flow channel 22, via pressure transmission interface 24, to pressure sensor 21, yet prevents the gas/air volume from transmitting a relatively high pressure from flow channel 22 to pressure sensor 21. The relatively high pressure may be any pressure that is greater than between approximately ten psi and approximately 100 psi, preferably greater than approximately fifty psi, for example, the aforementioned injection pressures, which can be up to 1200 psi. The volume of air/gas is preferably no greater than approximately ten cubic millimeters. Such a volume of air/gas does not significantly impact a compliance of the fluid circuit 200 downstream of pressure sensor 21 to degrade the frequency response of sensor 21 when monitoring blood pressure. It should be noted that a configuration of first part 23A of cavity 23 allows uninhibited movement of interface 24 at the lower pressures, to transmit the lower pressures to sensor 21 for blood pressure monitoring, as will be described in greater detail below.

With further reference to FIG. 2, double dashed lines are representative of interface 24 when collapsed into contact with a floor 235 of first part 23A of cavity 23, in response to the aforementioned relatively high pressure in flow channel 22; the collapsed interface 24 protects sensor 21 from exposure to the relatively high pressure. When the pressure in flow channel 22 subsides below the relatively high pressure, interface 24 rebounds away from floor 235, to again become operable for the transmission of patient blood pressures. According to preferred embodiments, a ratio of a volume of first part 23A of cavity 23 to a volume of second part 23B is between approximately one and six.

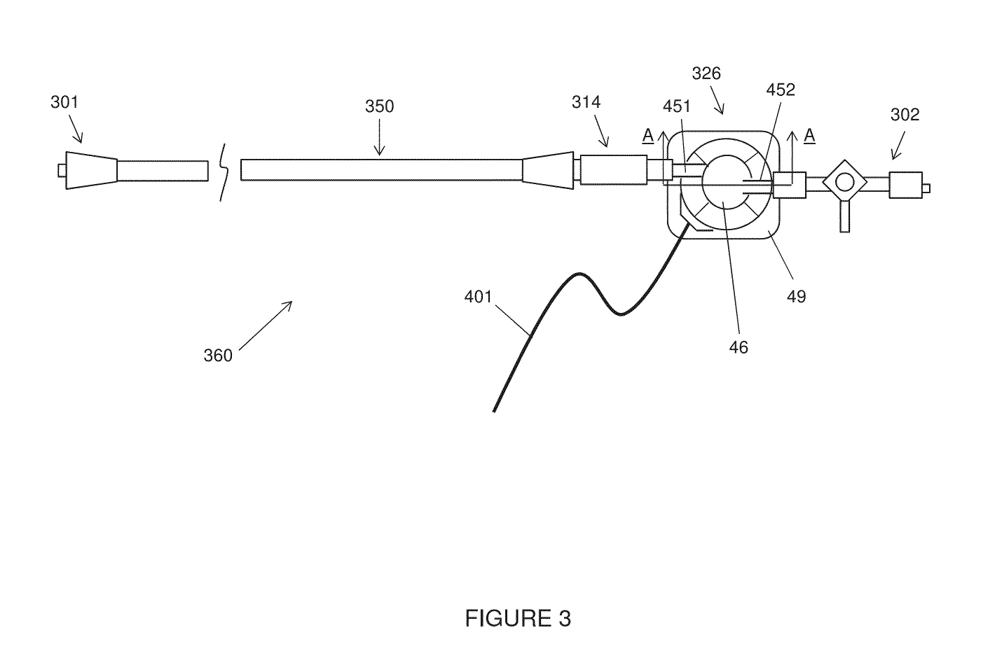

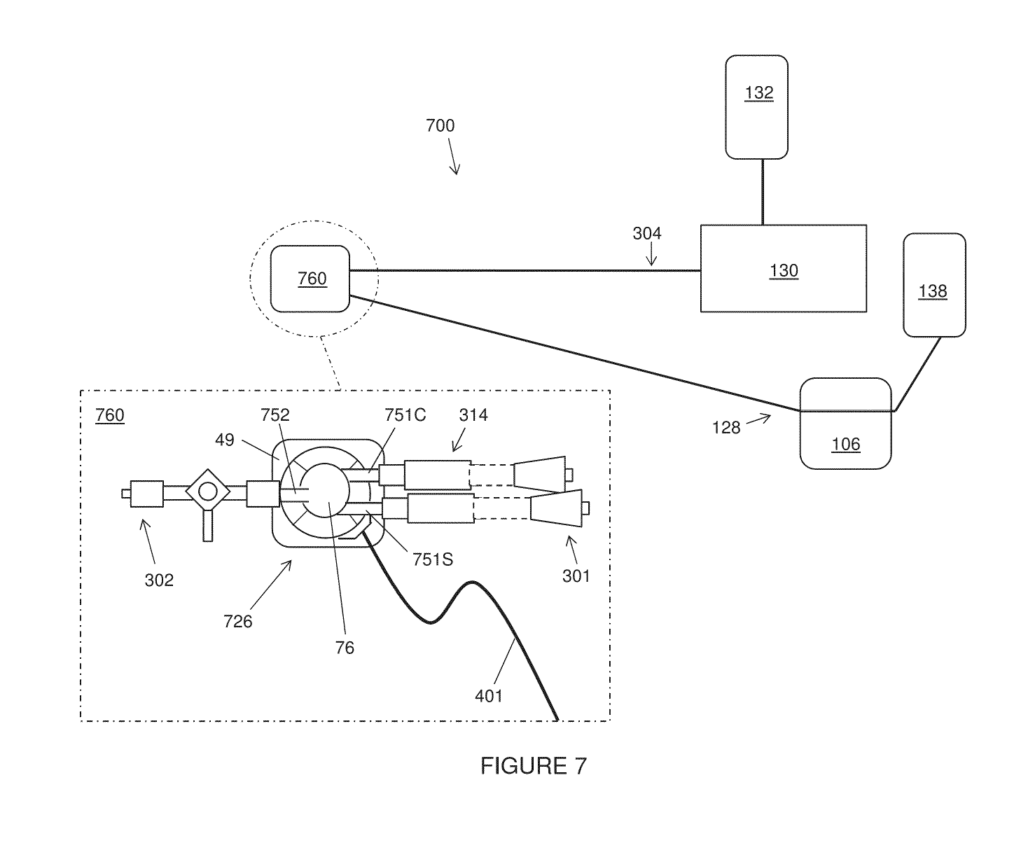

FIG. 3 is a plan view of a disposable fluid circuit subassembly 360, according to some embodiments, for example, which may be packaged as a kit and may be incorporated into injection system fluid circuit 200 of FIG. 2. FIG. 3 illustrates fluid circuit subassembly/kit 360 including a tubing line 350, that generally corresponds to line 122 of circuit 200, a pressure transducer assembly 326, that generally corresponds to assembly 226 of circuit 200, a check valve 314, which is coupled between assembly 326 and tubing line 350, and upstream and downstream connectors (e.g. Luer type fittings), 301 and 302, respectively, which are located at either end of subassembly 360. Subassembly 360 may be integrated into fluid circuit 200, being connected by upstream connector 301 to a y-junction 224, downstream of the convergence of lines 304 and 128. According to some alternate embodiments, one of which is described below in conjunction with FIG. 7, y-junction 224 and line 122/350 may be eliminated from fluid circuit 200.

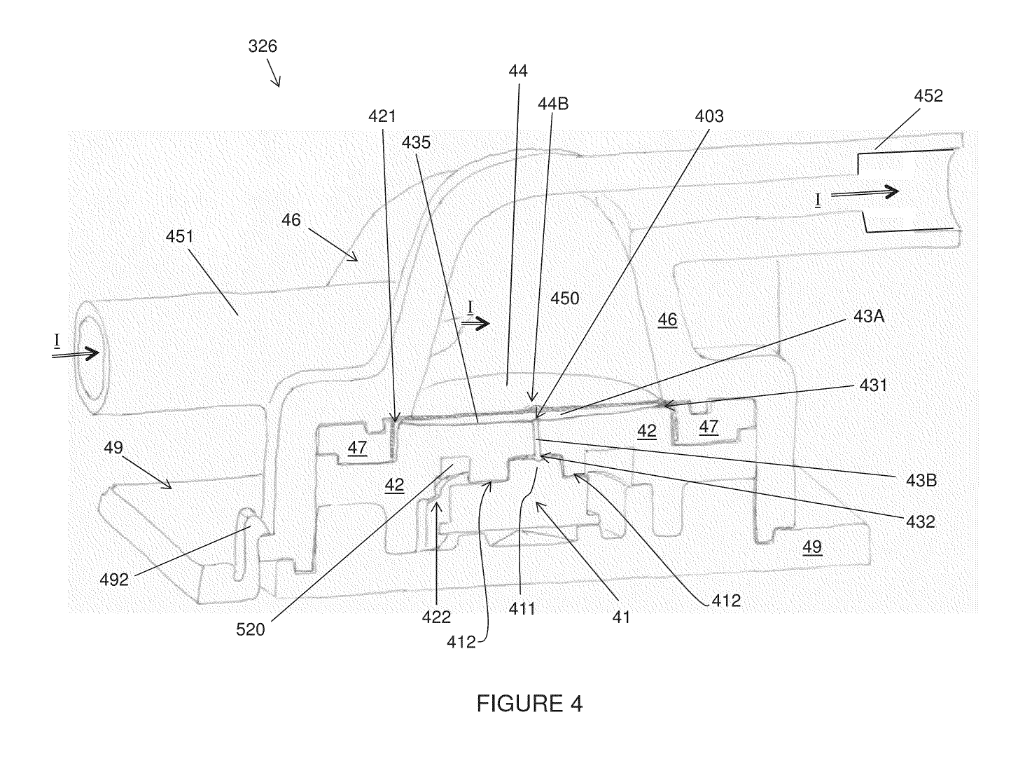

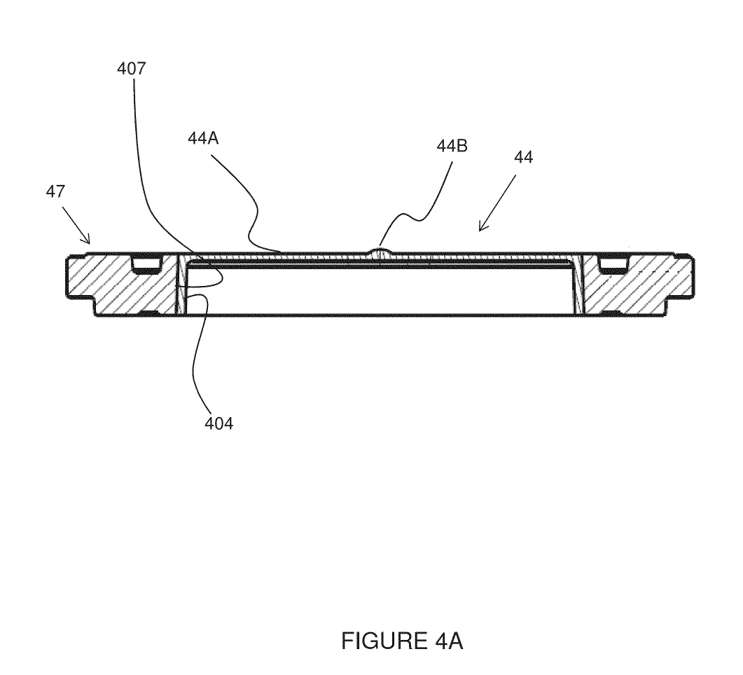

FIG. 4 is a cross-section view through section line A-A of FIG. 3, according to some embodiments. FIG. 4 illustrates pressure transducer assembly 326 including a housing 42 having a first side 421 coupled to a flow channel 450 of fluid circuit subassembly 360, and a second side 422, into which a pressure sensor 41 is mounted, and to which a base 49 is attached. Pressure sensor 41 will be described in greater detail below. Flow channel 450 is shown extending through a cap 46 of assembly 326 via an inlet port 451 and an outlet port 452, and the configuration of channel 450 within cap 46 will also be described in greater detail below. FIG. 4 further illustrates first and second parts, 43A and 43B, respectively, of a cavity, which is formed in housing 42 and is similar to cavity 23, described above. A pressure transmission interface 44 extends completely over a first opening 431 of the cavity, to isolate the cavity from flow channel 450, and pressure sensor 41 is located adjacent to a second opening 432 of the cavity, in fluid communication with the cavity. According to the illustrated embodiment, a gas volume fills an entirety of the cavity, and interface 44, for example, a flexible gas-permeable membrane, is responsive to pressures in flow channel 450, similar to interface 24, described above.

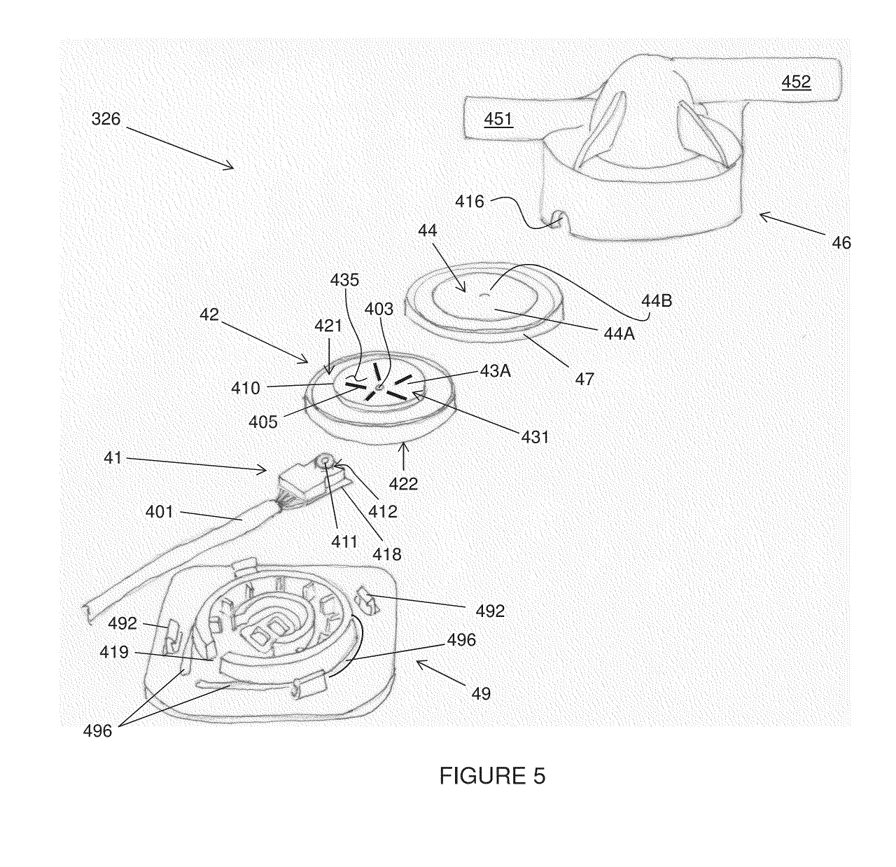

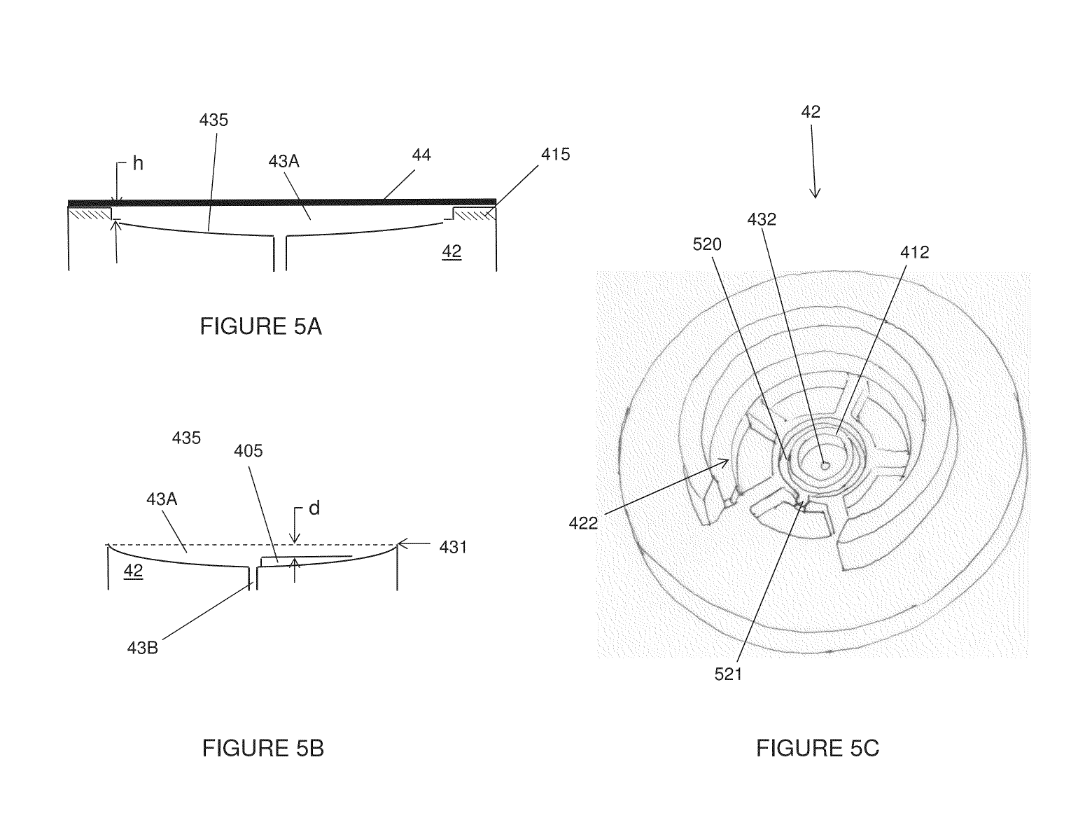

With further reference to FIG. 4, in conjunction with FIG. 5, which is an exploded view of pressure transducer assembly 326, according to some embodiments, cavity first part 43A is configured as a relatively shallow concavity, or depression in first side 421 of housing 42 and cavity second part 43B extends as a bore, from an opening 403, in a floor 435 of cavity first part 43A, to second opening 432 at second side 422 of housing 42, adjacent to pressure sensor 41. A contour of floor 435 of cavity first part 43A may follow a parabolic function and may have a maximum depth, at bore opening 403, relative to first opening 431, of approximately 0.006 inch.

According to the illustrated embodiment, interface 44 deflects into cavity first part 43A, for example, over a range of approximately 0.001 inch to approximately 0.002 inch, in response to relatively low pressures in flow channel 450, in order to transmit a patient's blood pressure signals, through the gas volume in the cavity, to pressure sensor 41. However, when pressures in flow channel 450 become relatively high, for example, greater than approximately ten to 100 psi and up to approximately 1200 psi during the aforementioned injections, interface 44 collapses into contact with floor 435, without penetrating into cavity second part 43B, to prevent transmission of pressures that could damage sensor 41. When pressures in flow channel 450 subside back into the lower range, interface 44 rebounds back out of contact with floor 435 and is, again, responsive to transmit the lower pressures to pressure sensor 41. With further reference to FIG. 5, according to some alternate embodiments, a perimeter edge 410 of floor 435 defines a step 415, for example, as shown in the schematic cross-section of FIG. 5A (cross-hatched). According to the illustrated embodiment, step 415 has a height h of approximately 0.003 inch, may act like a hinge for interface 44, and may prevent interface 44 from sticking to floor 435 after the relatively high pressures subside. In any case, both floor 435, and the facing side of interface 44 preferably have a matte finish to discourage adhesion therebetween.

FIG. 5 illustrates floor 435 of cavity first part 43A including one or more, optional, radially extending rib-like protrusions 405 formed therein. The one or more protrusions 405 may be useful in extending the life of assembly 326 if interface 44 becomes “somewhat flaccid” after aging or exposure to relatively high temperatures or other environmental conditions. FIG. 5B is a schematic cross-section through a portion of housing 42 to show a profile/elevation of one of the optional rib-like protrusions 405. According to some embodiments, a distance d between an interface-facing surface of each optional protrusion 405 and the plane of opening 431 (shown with a dashed line) is approximately 0.003 inch, so that, if the “somewhat flaccid” interface sags into cavity first part 43A, the one or more optional protrusions 405 can prevent interface 44 from blocking opening 403 into cavity second part 43B, when responding to lower pressures, for example, during blood pressure monitoring. Some embodiments may include the above-described step 415, defined around perimeter 410, in combination with one or more protrusions 405.

It should be noted that, if interface 44 is not “somewhat flaccid” the deflection thereof in response to relatively low pressures will not bring interface 44 into contact with the one or more optional protrusions 405. However, according to some alternate embodiments, the surfaces of protrusions 405 may be approximately flush with the plane of opening 431 to partially support interface 44 even when interface 44 had not become “somewhat flaccid”. It should be noted that a fewer number of protrusions 405, than that shown in FIG. 5, may be included in some embodiments. Furthermore, protrusions 405 may have a different form/geometry than that illustrated (rib-like), for example, protrusions 405 may be formed as spherical bumps in floor 435. In any case, the surfaces of the one or more protrusions 405 that come into contact with interface 44, either when interface 44 collapses under the relatively high pressures, or when the “somewhat flaccid” interface sags, are preferably rounded in order to prevent damage to interface 44.

FIGS. 4 and 5 further illustrate a ring 47 coupled about a perimeter of pressure transmission interface 44 and fitted within a groove of housing 42 to be sandwiched between housing 42 and cap 46, in order to secure interface 44 over first opening 431 of the cavity. The illustrated junction between housing 42 and cap 46 will be described below. According to some preferred embodiments, a diameter of first opening 431 is between approximately 8 mm and approximately 20 mm, and interface 44 is formed by a molded silicone rubber diaphragm, which has diameter that matches the diameter of opening 431, and a thickness, for example, between approximately 0.006 inch (0.15 mm) and approximately 0.012 inch (0.3 mm), to generally conform with floor 435 when collapsed under the aforementioned relatively high pressures. The silicone rubber diaphragm interface 44 may be insert-molded, according to methods known in the art, to ring 47, which is preferably formed from a polycarbonate, for example, APEC®1745, which is known in the art. With reference to FIG. 4A, an outer perimeter edge 404 of interface 44 is shown attached to an inner surface 407 of ring 47 by the insert molding process. Silicone rubber provides a suitable gas-permeable membrane, which allows for effective EtO sterilization of assembly 326, and for pressure sensor equilibration to local atmospheric pressure. According to an exemplary embodiment, 917CK silicone rubber (Minnesota Rubber & Plastics of Minneapolis, Minn.), which is preferably natural/translucent and has a durometer in the range of approximately 40-55, on a Shore A scale, forms pressure transmission interface 44. Although we have found minimal pressure errors, within an acceptable range (i.e. greater of ±3% of range or ±3 mmHg), for a durometer of approximately 50A, a lower durometer, for example, approximately 40A, may reduce pressure errors.

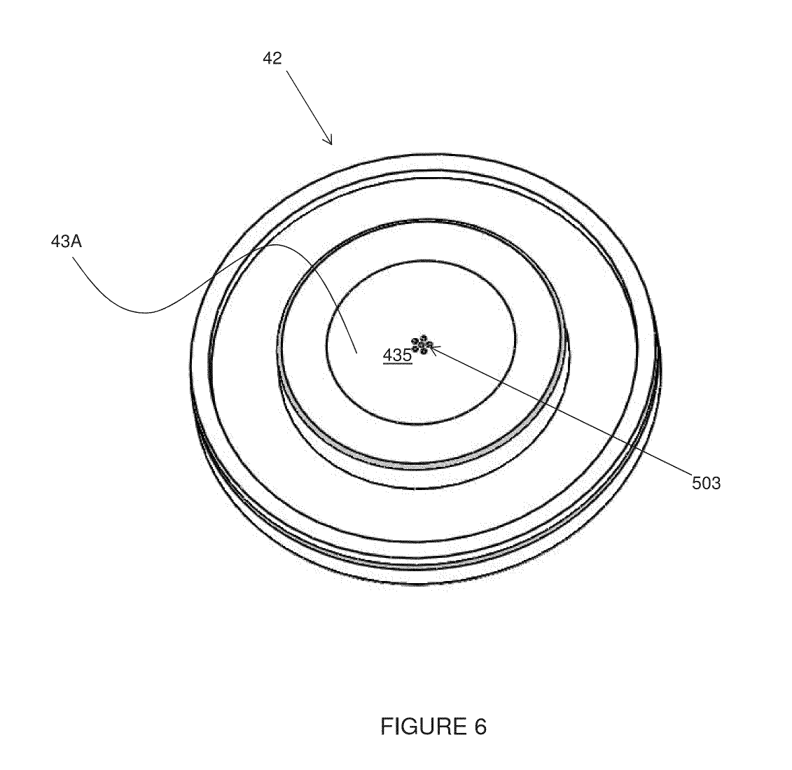

With further reference to FIGS. 4 and 5, a diameter of opening 403 is preferably smaller than a thickness of central zone 44B of the silicone rubber diaphragm interface 44, which is aligned over opening 403, so as to prevent interface 44 from penetrating/extruding into cavity second part 43B under the relatively high pressures. The thicker central zone 44B may be formed by a gate artifact of the silicone rubber molding process. According to exemplary embodiments, the diameter of opening 403, and cavity second part 43B, is between approximately 0.005 inch (0.13 mm) and approximately 0.015 inch (0.38 mm); and an edge of opening 403 is preferably rounded to prevent damage to interface 44 when collapsed against floor 435. According to some alternate embodiments, cavity second part 35B extends from a plurality of openings 503 in floor 435 of cavity first part 43A, for example, as illustrated in FIG. 6. A diameter of each of the plurality of openings 503 may be anywhere from about 0.001 inch to about 0.006 inch, for example, being formed by a laser trim, according to methods known in the art, and the number of openings 503 may be reduced or increased from that illustrated in FIG. 6.

According to some additional alternate embodiments, interface 44 may be constructed to have a stiffer central zone 44B, relative to perimeter zone 44A, without relying on the aforementioned increased thickness, for example, so that the diameter of opening 403 need not be less than a thickness of central zone 44B. According to an exemplary embodiment, central zone 44B of interface 44 may be formed as a disc from a polycarbonate, preferably the aforementioned APEC®1745, and perimeter zone 44A of interface 44 may be formed from a silicone rubber, which is, for example, over-molded onto the polycarbonate disc, according to methods known in the art. The polycarbonate disc that forms central zone 44B that may have undercuts for mechanical interlocking with the over-molded rubber.

With further reference to FIG. 5, pressure sensor 41 is shown including a gel interface 411 that overlays a measurement chip of sensor 41 and protrudes out therefrom. Such a construction is employed in the Model 1620 pressure transducer available from Measurement Specialties, Inc., and in the NPC-100 pressure sensor available from GE Novasensor, Inc. With reference back to FIG. 4, sensor 41 is shown fitted into second side 422 of housing 42 such that gel interface 411 is in close proximity to second opening 432 of the cavity. In some instances, gel interface 411 includes a meniscus that lies adjacent to opening 432 in assembly 326, and may increase the volume of cavity second part 43B by up to approximately two cubic millimeters. FIG. 5 further illustrates sensor 41 including a circuit board 418 on which the measurement chip is mounted and to which lead wires 401 are coupled for electrical connection of sensor 41 to a pressure monitoring system. According to an exemplary embodiment, sensor 41 is bonded and sealed to housing 42 with a bead of gel cyanoacrylate adhesive, for example, Loctite®4541™, that extends around a perimeter of gel interface 411, for example in the general area designated with reference numeral 412 in FIGS. 4, 5, and 5C. Cyanoacrylate adhesive provides a relatively quick partial cure (i.e. in 2-10 seconds) and then continues to cure over the next several hours; and the gel formulation of this adhesive keeps the adhesive in place until sensor 41 is fitted into second side 422. It should be noted that other types of adhesives, such as an UV curable adhesive, could be employed instead of cyanoacrylate. With reference to FIG. 5C, which shows second side 422 of housing 42, a moat 520 extends around area 412, according to some preferred embodiments, to receive excess adhesive flow when sensor 41 is fitted into second side 422. FIG. 5C further illustrates a drainage channel 521 extending from moat 520, to further direct excess uncured adhesive away from opening 432.

When all the illustrated parts of assembly 326 are coupled together, lead wires 401 of sensor 41 extend through a channel 419 of base 49 and a corresponding opening 416 of cap 46. An underside of base 49 preferably includes an adhesive surface (not shown) that temporarily attaches assembly 326 to the patient during a medical imaging procedure, for example, to drapes that cover the patient in proximity to the vascular access site. FIGS. 4-5 further illustrate base 49 of assembly 326 including detent type features 492 by which cap 46 is attached thereto, via interlocking engagement, after housing 42 and pressure transmission interface 44 are coupled to cap 46. As previously described and shown in FIG. 4, ring 47, to which interface 44 is coupled, is sandwiched between cap 46 and housing 42, and cap 46, ring 47 and housing 42 are all coupled together in any suitable manner, known to those skilled in the art, to provide a leak-tight seal therebetween that can withstand the aforementioned relatively high pressures, i.e. injections pressures up to 1200 psi. According to the illustrated embodiment, the mating interfaces between cap 46, ring 47 and housing 42 are configured to facilitate coupling by ultrasonic welding, according to methods known in the art, wherein interface 44 is isolated from the ultrasonic energy during the welding. With further reference to FIGS. 4 and 5, the interlocking engagement between cap 46 and base 49 is such that a groove 496 in base 49 engages a perimeter edge of cap 46 to prevent lateral expansion thereof, during high pressure injections, that could result in undue stresses on weld joints between cap 46 and ring 47 and between ring 47 and housing 42. FIG. 5 further illustrates optional external ribs (which may be increased in number) positioned about a perimeter of cap 46, for example, to provide additional support for weld joints. According to an exemplary embodiment, cap 46, housing 42 and base 49 are all formed, for example, by injection molding, from a relatively rigid plastic, preferably the aforementioned preferred polycarbonate (APEC®1745) that forms ring 47, for ultrasonic welding compatibility.

With further reference to FIG. 4, flow channel 450 is expanded within a flow chamber formed in cap 46, between inlet and outlet ports 451, 452. According to the illustrated embodiment, the chamber formed in cap 46 has a round cross section, in a plane approximately parallel to the flow through the ports 451, 452 (designated with arrow I), and an offset of inlet port 451 from a center-line of the round cross-section (best seen in FIG. 3) directs the flow from inlet port 451 tangentially, along an inner perimeter wall of the flow chamber. FIG. 4 further illustrates outlet port 452 of the chamber approximately aligned with the center-line of the aforementioned round cross-section of the chamber. The illustrated configuration of the flow chamber and ports results in swirling flow through the chamber, that may sweep up any air bubbles trapped along the wall of the flow chamber, for example, during pre-procedure purging of fluid circuit 200. According to some embodiments, LED lighting, for example, incorporated into a chip, which is either mounted alongside sensor 41 in second side 422 of housing 42, or integrated into circuit board 418 of sensor 41, may be employed in assembly 326 to illuminate the chamber portion of flow channel 450 for air bubble detection.

FIG. 7 is a block diagram of an alternate configuration of an injection system fluid circuit 700 with an enlarged plan view of a subassembly 760 thereof, according to some alternate embodiments. Like subassembly 360, introduced above in conjunction with FIG. 3, subassembly 760 may be disposable and packaged as a kit for incorporation into circuit 700, which includes, like circuit 200, a first fluid reservoir 132 that supplies a pressurizing unit 130 for injection of, for example, a radiopaque contrast agent, into a patient's vascular system via a fluid circuit line 304, and a second fluid reservoir 138 from which a diluent, such as saline, is drawn by a peristaltic pump 106 through another fluid circuit line 128 for injection. FIG. 7 illustrates subassembly 760 including upstream and downstream connectors 301, 302, and a pressure transducer assembly 726, which, according to preferred embodiments, shares a cross-section with the above-described pressure transducer assembly 326 (indicated with section line A-A in FIG. 3 and shown in FIG. 4), as well as almost all of the components of assembly 326. Assembly 726 differs from assembly 326 in that a cap 76 of assembly 726 is formed with a pair of inlet ports 751C and 751S, each of which has an upstream connector 301 coupled thereto for the purpose of connecting a flow chamber (similar to flow channel 450 in FIG. 4) that is formed within cap 76 directly to lines 304 and 128, without requiring a y-connector and an added length of fluid line, for example, y-connector 224 and line 122 shown in FIG. 2. Since saline (from reservoir 138) may be directly flushed through the flow chamber formed in cap 76, at port 751S, after a contrast injection through port 751C, the fluid between the patient's vascular system and the pressure sensor of assembly 726 may have a lower viscosity (less residual contrast and more saline), resulting in greater signal fidelity for improved pressure monitoring. Each of inlet ports 751C, 751S is preferably offset from a center-line of the round cross-section of the flow chamber formed within cap 76 to direct flow tangentially along an inner perimeter wall of the flow chamber, for swirling flow like that described above for cap 46. Dashed lines in the enlarged plan view of FIG. 7 represent optional lengths of tubing extending between check valves 314 and connectors 301.

In the foregoing detailed description, the invention has been described with reference to specific embodiments. However, it may be appreciated that various modifications and changes can be made without departing from the scope of the invention as set forth in the appended claims.

We claim:

1. A pressure transducer assembly for a medical injection system, the assembly comprising:
a pressure sensor;
a housing including a first side and a second side, the first side being coupled to a flow channel of a fluid circuit for the injection system, and the pressure sensor being assembled into the second side;
a cavity formed within the housing and extending from a first opening thereof at the first side of the housing to a second opening thereof adjacent to the second side of the housing, the pressure sensor being located adjacent to the second opening of the cavity and in fluid communication with the cavity;
a pressure transmission interface extending completely over the first opening of the cavity and isolating the cavity from the flow channel of the injection system; and
a gas volume filling an entirety of the cavity;
wherein a configuration of the cavity allows the gas volume to transmit a patient's blood pressure from the flow channel, via the pressure transmission interface, to the pressure sensor, yet prevents the gas volume from transmitting a relatively high pressure from the flow channel, via the pressure transmission interface, to the pressure sensor, the relatively high pressure being any pressure greater than between approximately 10 psi and approximately 100 psi.

2. The assembly of claim 1, wherein the relatively high pressure is any pressure greater than approximately 50 psi.

3. The assembly of claim 1, wherein the gas volume is no greater than approximately ten cubic millimeters.

4. The assembly of claim 1, wherein:
the cavity comprises a first part adjacent to the pressure transmission interface and a second part adjacent to the pressure sensor; and
a ratio of a volume of the first part to a volume of the second part is between approximately one and approximately six.

5. The assembly of claim 1, wherein the cavity comprises a first part adjacent to the pressure transmission interface and a second part adjacent to the pressure sensor, the first part having a floor that the pressure transmission interface abuts at the relatively high pressure, and the second part extending from an opening in the floor to the second opening of the cavity.

6. The assembly of claim 5, wherein the second part of the cavity comprises a bore having a diameter between approximately 0.005 inch (0.13 mm) and approximately 0.015 inch (0.38 mm).

7. The assembly of claim 5, wherein the floor of the first part of the cavity includes a plurality of openings from which the second cavity extends.

8. The assembly of claim 1, wherein the pressure transmission interface comprises a flexible gas-permeable membrane.

9. The assembly of claim 8, wherein the membrane includes a central zone and a perimeter zone, the central zone being stiffer than the perimeter zone.

10. The assembly of claim 9, wherein:
the cavity comprises a first part adjacent to the pressure transmission interface and a second part adjacent to the pressure sensor, the second part extending from an opening in a floor of the first part of the cavity to the second opening of the cavity; and
the central zone of the membrane is approximately aligned over the opening in the floor of the first part of the cavity.

11. The assembly of claim 1, wherein the pressure transmission interface comprises a silicone rubber diaphragm.

12. The assembly of claim 11, wherein the diaphragm has a diameter between approximately 8 mm and approximately 20 mm.

13. The assembly of claim 11, wherein an average thickness of the diaphragm is between approximately 0.006 inch (0.15 mm) and approximately 0.012 inch (0.3 mm).

14. The assembly of claim 11, wherein the diaphragm includes a central zone and a perimeter zone, the central zone having a thickness greater than that of the perimeter zone.

15. The assembly of claim 11, wherein the diaphragm includes a central zone and a perimeter zone, the central zone being stiffer than the perimeter zone.

16. The assembly of claim 15, wherein:
the cavity comprises a first part adjacent to the pressure transmission interface and a second part adjacent to the pressure sensor, the second part extending from an opening in a floor of the first part of the cavity to the second opening of the cavity; and
the central zone of the diaphragm is approximately aligned over the opening in the floor of the first part of the cavity.

17. The assembly of claim 16, wherein a thickness of the central zone of the diaphragm is greater than a diameter of the opening in the floor.

18. The assembly of claim 1, wherein:
the cavity comprises a first part adjacent to the pressure transmission interface and a second part adjacent to the pressure sensor, the second part extending from an opening in a floor of the first part of the cavity to the second opening of the cavity; and
the pressure transmission interface moves into contact with the floor of the first part of the cavity at the relatively high pressure and rebounds back out of contact with the floor when pressures subside below the relatively high pressure.

19. The assembly of claim 1, wherein the cavity comprises a first part adjacent to the pressure transmission interface and a second part adjacent to the pressure sensor, the first part having a concave floor that the pressure transmission interface abuts at the relatively high pressure, and the second part extending from an opening in the floor to the second opening of the cavity.

20. The assembly of claim 19, wherein a maximum distance between the concave floor and the first opening of the cavity is approximately 0.006 inch.

21. The assembly of claim 19, wherein the concave floor of the first part of the cavity includes at least one radially extending rib-like protrusion formed therein, a distance between a surface of the at least one protrusion, which faces the interface, and the first opening of the cavity being approximately 0.003 inch.

22. The assembly of claim 1, further comprising a cap in which a flow chamber for the flow channel is formed, the cap including an inlet port and an outlet port, and the cap being coupled to the first side of the housing around a perimeter of the pressure transmission interface.

23. The assembly of claim 22, wherein the flow chamber has a round cross-section, in a plane approximately parallel to flow through the ports, and the inlet port is offset from a center-line of the round cross-section at a location to direct the flow tangentially from the inlet port along an inner perimeter wall of the flow chamber.

24. The assembly of claim 23, wherein the outlet port of the flow chamber is approximately aligned with the center-line of the round cross-section of the flow chamber.

25. The assembly of claim 22, wherein the pressure transmission interface comprises a flexible gas-permeable membrane and the perimeter thereof is secured between the cap and the housing.

26. The assembly of claim 1, further comprising a cap in which a flow chamber for the flow channel is formed, the cap being coupled to the first side of the housing around a perimeter of the pressure transmission interface; and wherein the flow channel of the fluid circuit comprises a pair of lines and the cap includes a corresponding pair of inlet ports and an outlet port.

27. The assembly of claim 1, further comprising a flow chamber adjacent to the first side of the housing, the flow chamber including inlet and outlet ports for integration into the flow channel, and the pressure transmission interface extending directly between the flow chamber and the cavity.

28. A method for isolating a pressure sensor in a fluid circuit of a medical injection system during high pressure injection flow through the fluid circuit, the pressure sensor being active for blood pressure monitoring prior to, and/or following the high pressure injection flow, and the method comprising:
enclosing a volume of air between the pressure sensor and a first side of a flexible gas-permeable membrane, the volume of air being enclosed in a cavity in fluid communication with the pressure sensor and the first side of the membrane; and
coupling the enclosed volume of air to the fluid circuit such that a second, opposite side of the membrane is exposed to the flow through the fluid circuit;
wherein the cavity is configured to allow the volume of air to transmit a patient's blood pressure from the fluid circuit, via the membrane, to the pressure sensor, yet prevents the volume of air from transmitting pressures of the high pressure injection flow, via the membrane, to the pressure sensor, the pressures of the high pressure injection flow being greater than between approximately 10 psi and approximately 100 psi.

29. The method of claim 28, further comprising:
forming the flexible gas permeable membrane from silicone rubber; and
securing a perimeter of the membrane between a housing and a cap;
wherein the housing includes the cavity formed therein and has the pressure sensor mounted thereto; and
the cap includes a flow chamber, an inlet port, and an outlet port, the inlet and outlet ports coupling the flow chamber to the fluid circuit so that the high pressure injection flow passes therethrough.

30. A disposable fluid circuit kit for a medical injection system comprising a pressure transducer assembly, an upstream connector and a downstream connector; and the pressure transducer assembly comprising:
a housing;
a pressure sensor mounted to the housing;
a cavity formed within the housing and extending from an opening thereof to the mounted pressure sensor so that the sensor is in fluid communication with the cavity;
a flow chamber adjacent to the opening of the cavity, the flow chamber having an inlet port coupled to the upstream connector and an outlet port coupled to the downstream connector;
a gas volume filling an entirety of the cavity; and
a pressure transmission interface extending between the cavity and flow chamber and completely over the opening of the cavity to isolate the cavity from the flow chamber and to enclose the gas volume within the cavity;
wherein a configuration of the cavity allows the gas volume to transmit a patient's blood pressure from the flow chamber, via the pressure transmission interface, to the pressure sensor, yet prevents the gas volume from transmitting a relatively high pressure from the flow chamber, via the pressure transmission interface, to the pressure sensor, the relatively high pressure being any pressure greater than between approximately 10 psi and approximately 100 psi.

31. The kit of claim 30, wherein the pressure is any pressure greater than approximately 50 psi.

32. The kit of claim 30, wherein the gas volume of the pressure transducer assembly is no greater than approximately ten cubic millimeters.

33. The kit of claim 30, wherein:
the cavity of the pressure transducer assembly comprises a first part adjacent to the pressure transmission interface of the assembly and a second part adjacent to the pressure sensor of the assembly; and
a ratio of a volume of the first part to a volume of the second part is between approximately one and approximately six.

34. The kit of claim 30, wherein the cavity of the pressure transducer assembly comprises a first part adjacent to the pressure transmission interface of the assembly and a second part adjacent to the pressure sensor of the assembly, the first part having a floor that the pressure transmission interface abuts at the relatively high pressure, and the second part extending from an opening in the floor to the pressure sensor.

35. The kit of claim 34, wherein the second part of the cavity comprises a bore having a diameter between approximately 0.005 inch (0.13 mm) and approximately 0.015 inch (0.38 mm).

36. The kit of claim 34, wherein the floor of the first part of the cavity includes a plurality of openings from which the second cavity extends.

37. The kit of claim 30, wherein the pressure transmission interface of the pressure transducer assembly comprises a flexible gas-permeable membrane.

38. The kit of claim 37, wherein the membrane includes a central zone and a perimeter zone, the central zone being stiffer than the perimeter zone.

39. The kit of claim 38, wherein:
the cavity of the pressure transducer assembly comprises a first part adjacent to the pressure transmission interface of the assembly and a second part adjacent to the pressure sensor of the assembly, the second part extending from an opening in a floor of the first part of the cavity to the pressure sensor; and
the central zone of the membrane is approximately aligned over the opening in the floor of the first part of the cavity.

40. The assembly of claim 30, wherein the pressure transmission interface of the pressure transducer assembly comprises a silicone rubber diaphragm.

41. The kit of claim 40, wherein the diaphragm has a diameter between approximately 8 mm and approximately 20 mm.

42. The kit of claim 40, wherein an average thickness of the diaphragm is between approximately 0.006 inch (0.15 mm) and approximately 0.012 inch (0.3 mm).

43. The kit of claim 40, wherein the diaphragm includes a central zone and a perimeter zone, the central zone having a thickness greater than that of the perimeter zone.

44. The assembly of claim 40, wherein the diaphragm includes a central zone and a perimeter zone, the central zone being stiffer than the perimeter zone.

45. The kit of claim 44, wherein:
the cavity of the pressure transducer assembly comprises a first part adjacent to the pressure transmission interface of the assembly and a second part adjacent to the pressure sensor of the assembly, the second part extending from an opening in a floor of the first part of the cavity to the pressure sensor; and
the central zone of the diaphragm is approximately aligned over the opening in the floor of the first part of the cavity.

46. The kit of claim 45, wherein a thickness of the central zone of the diaphragm is greater than a diameter of the opening in the floor.

47. The kit of claim 30, wherein:
the cavity of the pressure transducer assembly comprises a first part adjacent to the pressure transmission interface of the assembly and a second part adjacent to the pressure sensor of the assembly, the second part extending from an opening in a floor of the first part of the cavity to the pressure sensor; and
the pressure transmission interface moves into contact with the floor of the first part of the cavity at the relatively high pressure and rebounds back out of contact with the floor when pressures subside below the relatively high pressure.

48. The kit of claim 30, wherein the cavity of the pressure transducer assembly comprises a first part adjacent to the pressure transmission interface of the assembly and a second part adjacent to the pressure sensor of the assembly, the first part having a concave floor that the pressure transmission interface abuts at the relatively high pressure, and the second part extending from an opening in the floor to the pressure sensor.

49. The kit of claim 48, wherein a maximum distance between the concave floor and the opening of the cavity is approximately 0.006 inch.

50. The kit of claim 48, wherein the concave floor of the first part of the cavity includes at least one radially extending rib-like protrusion formed therein, a distance between a surface of the at least one protrusion, which faces the interface, and the opening of the cavity being approximately 0.003 inch.

51. The kit of claim 30, wherein the flow chamber of the pressure transducer assembly has a round cross-section in a plane approximately parallel to flow through the inlet port of the flow chamber, and the inlet port is offset from a center-line of the round cross-section at a location to direct the flow tangentially from the inlet port along an inner perimeter wall of the flow chamber.

52. The kit of claim 51, wherein the outlet port of the flow chamber is approximately aligned with the center-line of the round cross-section of the flow chamber.

53. The kit of claim 30, wherein the upstream connector is one of a pair of upstream connectors of the kit, and the flow chamber of the pressure transducer assembly has a corresponding pair of inlet ports.
